(19) United States
(12) Patent Application Publication
(10) Pub. No.: US 2025/0114577 A1
(43) Pub. Date: Apr. 10, 2025

(54) Profiled Drug-Coated Balloons Using Nanoparticles and Methods of Drug Delivery Therewith

(71) Applicant: Advanced Nanotherapies, Inc., Cleveland, OH (US)

(72) Inventors: Marwan Berrada-Sounni, Los Gatos, CA (US); Francis Proch, Chardon, OH (US); Ryan Nowicki, Olmsted Twp, OH (US)

(21) Appl. No.: 18/729,706

(22) PCT Filed: Jan. 30, 2023
(86) PCT No.: PCT/US2023/011822

§ 371 Date: Jul. 17, 2024

Related U.S. Application Data
(60) Provisional application No. 63/304,347, filed on Jan. 28, 2022.

(51) Int. Cl. A61M 25/10; A61L 29/08; A61L 29/16

(52) U.S. Cl. CPC A61M 25/10184; A61L 29/085; A61L 29/16; A61L 2300/416; A61L 2420/06; A61M 2025/105; A61M 2025/1061

(57) ABSTRACT

Profiled drug-coated balloons for nanoparticle-based drug delivery having a reduced-diameter central balloon portion and methods of drug delivery using same are disclosed. The profiled drug-coated balloon may comprise: distal and proximal balloon end portions having a first nominal full inflation diameter; a central balloon portion between said end portions, the central balloon portion having a second, lesser nominal full inflation diameter; a drug carrying nanoparticle coating on said central balloon portion.

### RELATED APPLICATION DATA

This application claims the benefit of priority of U.S. Provisional Patent Application Ser. No. 63/304,347, filed Jan. 28, 2022, and titled “Profiled Drug-Coated Balloons Using Nanoparticles and Methods of Drug Delivery Therewith”, which is incorporated by reference herein in its entirety.

### FIELD

The present disclosure generally relates to drug-coated balloons and methods, and more specifically to profiled drug-coated balloons using nanoparticles and methods of drug delivery therewith.

### BACKGROUND

During vessel treatment with a drug-coated balloon (DCB), various phases occur: 1) a Transit phase with the balloon in a folded configuration and delivered to lesion site, followed by 2) an Inflation phase in which the balloon is held at the treatment location and contrast fluid is supplied to fill and expand the balloon, then 3) an Inflation-Hold phase during which full balloon expansion is reached and balloon/vessel opposition is held for a specified length of time, and lastly 4) a Removal phase during which the balloon is deflated and removed. Advantageous features in DCB design include configurations to minimize drug loss during the Transit and Inflation phases, and allow for maximum drug coating transfer during inflation in the Inflation-Hold phase.

In general, conventional DCBs utilize drugs in a crystalline form adhered to the balloon surface by various techniques. Because of the use of the crystalline form it is generally considered to be a best practice to provide substantially full contact between the coated surfaces of the DCB and the vessel wall in order to push the crystalline form of the drug against the vessel in a similar fashion for pushing barbs against the vessel wall.

However, when delivering drugs in other forms, for example with nanoparticles as a delivery vehicle, the substantially full contact model of conventional DCB designed for use with drugs in crystalline form may not provide maximum drug coating transfer due to differences in the transfer mechanism.

When using nanoparticles as a delivery vehicle for the drug in a drug-coated balloon (DCB) it may be desirable to first dissolve the nanoparticle coating on the DCB at the treatment location before attempting to drive the nanoparticles into the vessel tissue. Dissolving the nanoparticle coating can create a plume of nanoparticles in an aqueous fluid around the DCB. With such an aqueous nanoparticle-filled environment surrounding the DCB, a high-pressure environment between the vessel wall and the balloon can facilitate movement of the nanoparticle into the tissue. Conventional DCBs are not well-suited to creating such a high-pressure environment because the mechanism of delivery is focused on direct impingement of the balloon surface with the vessel wall to force the drug crystals into the tissue. However, in the case of nanoparticle delivery, the plume of nanoparticles as described above can be swept away in whole or in part by blood flow around the DCB as the nanoparticles dissolve from the surface. Alternatively, immediate engagement of the DCB with the vessel wall reduces exposure of the nanoparticles to the aqueous environment needed to dissolve the coating.

### SUMMARY

In one implementation, the present disclosure is directed to a profiled drug-coated balloon, which includes distal and proximal balloon end portions having a first nominal full inflation diameter; a central balloon portion between the end portions, the central balloon portion having a second, lesser nominal full inflation diameter; a drug carrying nanoparticle coating on the central balloon portion.

In another implementation, the present disclosure is directed to a method of intravascular drug delivering using a drug-coated balloon. The method includes providing a profiled balloon with enlarged distal and proximal end portions and a reduced-diameter central portion, wherein a drug carrying nanoparticle coating is provided on the reduced-diameter central portion; positioning the profiled balloon at an intravascular treatment site with the balloon in a deflated state; inflating the enlarged distal and proximal end portions to contact the vascular wall bounding the treatment site; and inflating the reduced-diameter central portion after the enlarged distal and proximal end portions contact the vascular wall.

In yet another implementation, the present disclosure is directed to a method of intravascular drug delivering using a drug-coated balloon. The method includes providing a profiled balloon with enlarged distal and proximal end portions and a reduced-diameter central portion, wherein a drug carrying nanoparticle coating is provided on the reduced-diameter central portion; positioning the profiled balloon at an intravascular treatment site with the balloon in a deflated state; inflating the enlarged distal and proximal end portions to contact the vascular wall bounding the treatment site; inflating the reduced-diameter central portion after the enlarged distal and proximal end portions contact the vascular wall; overinflating the reduced-diameter central portion to form a convex wall section; applying cycling pressure to the reduced-diameter central portion to create undulating pressure waves across the treatment site bounded by the enlarged distal and proximal balloon portions; and deflating and removing the balloon without first removing any excess drug compositions surrounding the balloon.

### BRIEF DESCRIPTION OF DRAWINGS

For the purpose of illustrating the disclosure, the drawings show aspects of one or more embodiments of the disclosure. However, it should be understood that the present disclosure is not limited to the precise arrangements and instrumentalities shown in the drawings, wherein:

FIG. 1 is a schematic cross-sectional view and detail of an embodiment of a profiled drug-coated balloon according to the present disclosure.

FIG. 2 is a detail view of an enlarged end portion of a profiled balloon according to a disclosed embodiment.

FIG. 3 is a schematic cross-sectional view of another embodiment of a profiled drug-coated balloon according to the present disclosure.

FIGS. 4A, 4B, 4C and 4D are sequential views of deployment, inflation and over-inflation for an embodiment of a profiled drug-coated balloon according to the present disclosure.

FIG. 5 is an enlarged partial schematic cross-sectional view of the profiled drug-coated balloon shown in FIG. 4D in an over-inflated state with the relative appearance of over-inflation exaggerated for illustration purposes.

### DETAILED DESCRIPTION

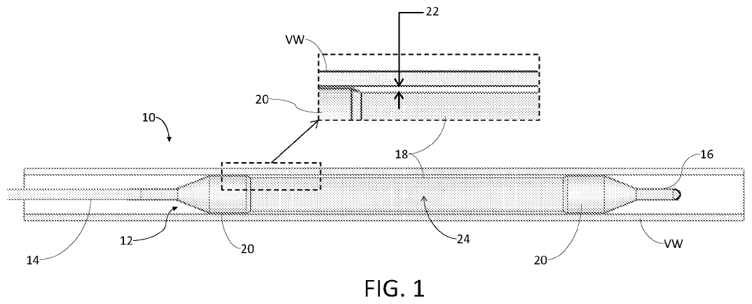

To address the unique requirements of nanoparticle-based drug delivery, embodiments of DCBs disclosed herein have a specific profile configured to create a reservoir for dissolving and exerting pressure on the nanoparticle plume after dissolving to force the drug-carrying nanoparticles into the surrounding tissue. Thus, in some embodiments, as illustrated in FIG. 1, DCB 10 generally includes a profiled balloon 12, a proximal inflation lumen 14 and distal end 16. Profiled balloon 12 is formed with a slight dumbbell or dog bone shape having a reduced-diameter central portion 18 and enlarged end portions 20. Enlarged end portions 20 are sized to contact the vessel wall (VW) at full inflation so as to create an isolated reservoir 22 surrounding central portion 18. Isolated reservoir 22 allows the creation of a high-pressure environment within the coating reservoir to allow nanoparticle rehydration and to facilitate nanoparticle-based drug delivery as described above.

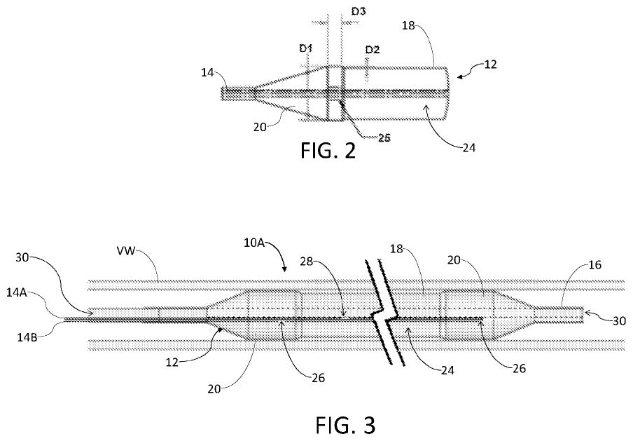

FIG. 2 shows an example of an enlarged end portion 20 of a profiled balloon 12 according to one embodiment of the present disclosure. As shown therein, the overall diameter DI of enlarged end portions 20 may range from about 1.5 mm to about 7.0 mm depending on specific balloon sizes. Reduced-diameter central portion 18, wherein nanoparticle coating 24 is provided, will have a diameter that is less than diameter D1 by dimension D2, with D2 typically being in the range of about 0.050 mm to 0.5 mm. A non-tapered section of end portion 20 may have a longitudinal length D3 in the range of about 0.1 to about 1.0 mm. To facilitate visualization, radiopaque marker rings 25 may be provided as shown or in other alternative locations as may be determined by persons skilled in the art. The length of central balloon portion 18 may be in the range of about 10 mm to about 200 mm depending on application. For example, for coronary applications, central portion 18 would typically be in the range of about 10-40 mm in length, whereas for peripheral artery applications central portion 18 would typically be in the range of about 40-200 mm in length.

In some embodiments, a drug-containing nanoparticle coating 24 on the surface of the reduced-diameter central portion 18 of profiled balloon 12 may be created using a layer-by-layer process. The total drug loading per balloon may be in the range of about 1.0 to about 3.5 μg/mm2 drug. As one illustrative example, with approximately sixteen (16) layers applied, the coating thickness is approximately 50 μm. To accommodate this example drug loading in reduced-diameter portion 18, that portion may have a nominal full inflation diameter approximately 100 μm less than enlarged ends 20. However, in some embodiments, the reduction in diameter in reduced-diameter portion 18 may be up to 1000 μm less than enlarged end portions 20.

Drug-containing nanoparticle coating 24 may comprise functionalized nanoparticles (f-NP), wherein f-NP is polymeric nanosphere, i.e., a matrix solid nanoparticle (not a polymeric micelle, liposome (organic) or dendrimer). The drug-containing nanoparticle coating provides high retention of drug on the DCB while permitting handling in an ordinary course and also promotes a high fraction of drug delivery without necessitating hydrophilic coating layers to retain the drug while facilitating handling. Drug formulations may comprise sirolimus, sirolimus derivative, paclitaxel or combinations thereof, and/or cytostatic/cytotoxic drug combinations in amorphous, non-crystalline form, contained within PLGA. Dual drug formulations of sirolimus and paclitaxel are in particular contemplated. In some embodiments, the nanoparticle coating may be provided in three layers on the balloon surface: (1) a base coat on the balloon of polyvinyl alcohol (PVA) which acts as an adhesive to retain nanoparticles, (2) a drug-loaded nanoparticle layer, and (3) optional outer PVA coating serving as a protective layer until the balloon is deployed in the vessel, whereupon hydration from contact with blood dissolves the outer PVA layer to allow contact of the nanoparticles with treatment site with expansion of balloon and the base coat PVA subsequently dissolves, also due to hydration, to release the nanoparticles. Further details of f-NP preparations are described, for example, in U.S. Patent Publication No. US20220193310A1, entitled “Dual agent nanoparticle composition for coating medical devices,” which is incorporated by reference in its entirety.

In further alternative embodiments balloon 12 is configured so that end portions 20 begin to inflate before central portion 18. In some embodiments, end portions 20 may inflate to engage the vessel wall before substantial inflation of central portion 18. In order to cause balloon end portions 20 to first inflate, the end portions may be configured with a thinner, more compliant material as compared to central portion 18. Thus as pressure is initially applied to the inflation media, end portions 20 will begin inflation at a lower pressure and central portion 18 will only begin to inflate once a higher pressure is reached. Persons of ordinary skill may configure the end portions and central portions based on the teachings herein to achieve sequential inflation as described with the central portion inflating at a predetermined threshold pressure. In alternative embodiments, sequential inflation as described may be configured by different sizing and positioning of inflation ports from the central inflation lumen into the different balloon portions. For example, flow restrictions may be used to delay inflation of central portion 18.

In a further alternative embodiment, shown in FIG. 3, DCB 10A is provided with inflation lumen 14A communicating with balloon central portion 18 via inflation ports 28, and with inflation lumen 14B communicating with end portions 20 via inflation ports 26. In this alternative embodiment, central guidewire lumen 30 is provided across profiled balloon member 12.

Using DCBs 10/10A according to the present disclosure, balloon 12 is inflated using conventional balloon inflation techniques and tools, such as an angioplasty inflator, etc. Due to configuration of balloon 12, proximal and distal end portions 20 of balloon 12 expand first, hence making vessel opposition before central portion 18. Such first step creates the coating reservoir with end portions 20 in some embodiments acting as a seal. As the inflation continues, lesser diameter central portion 18 also expands, increasing the pressure applied in reservoir 22 (similar in fashion to a plunger in a fixed volume) and any coating pre-releasing from the balloon is trapped in coating reservoir 22 and pressed into the vessel via hydraulic or mechanical pressure. Once full inflation is reached, the drug coating in the reservoir created by the stepped-down diameter central portion 18 also remains present during the inflation hold period, to further add to the coating total uptake.

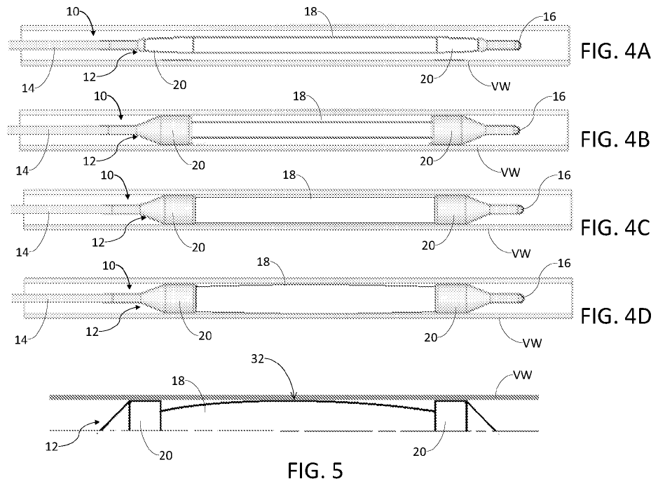

FIGS. 4A-D illustrate a further alternative embodiment of a drug-delivery method using a profiled balloon 12 as disclosed herein. In FIG. 4A, DCB 10 is initially positioned at an intravascular treatment site in fully deflated state. Once position is confirmed by conventional visualization techniques, enlarged end portions 20 are first inflated to engage the vessel wall as shown in FIG. 4B. Next, as shown in FIG. 4C, central portion 18 is fully inflated to its nominal diameter, defining reservoir 22 slightly spaced from vessel wall as described above (see FIG. 1). Nanoparticle hydration begins to form a drug/gel layer at this stage. Thereafter, as shown in FIG. 4D, central portion 18 is overinflated to dynamically drive hydrated gel layer into the surrounding tissue of the vessel wall. Use of a convex wall 32 created by over inflation, as shown schematically in FIG. 5, may be used as a further effect in driving drug-carrying nanoparticles into surrounding tissue of the vessel wall. Overinflation may be a single step or repeated process, and may include dynamic effects such as undulating pressure waves. Examples of systems for creating dynamic/undulating pressure waves are disclosed in Applicant's prior-filed International Publication Number WO 2021/178452 A3, entitled “Undulating Balloon Systems and Methods for Nanoparticle-based Drug Delivery,” which is incorporated by reference herein in its entirety.

Various modifications and additions can be made without departing from the spirit and scope of this disclosure. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present disclosure. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this disclosure or of the inventions as set forth in following claims.

What is claimed is:

1. A profiled drug-coated balloon, comprising:
distal and proximal balloon end portions having a first nominal full inflation diameter;
a central balloon portion between said end portions, the central balloon portion having a second, lesser nominal full inflation diameter;
a drug carrying nanoparticle coating on said central balloon portion.

2. The profiled drug-coated balloon of claim 1, further comprising at least one inflation lumen communicating with said balloon portions.

3. The profiled drug-coated balloon of claim 2, further comprising separate inflation lumens for the balloon end portions and central balloon portion.

4. The profiled drug-coated balloon of claim 1, wherein the first nominal full inflation diameter of the balloon end portions is in a range of about 1.5 mm to about 7.0 mm, and the second, lesser nominal full inflation diameter of the central balloon portion is about 0.05 mm to about 0.50 mm less than the first nominal full inflation diameter.

5. The profiled drug-coated balloon of claim 1, wherein said drug carrying nanoparticle coating comprises one or more of sirolimus, sirolimus derivative, paclitaxel or combinations thereof, cytostatic/cytotoxic drug combinations in amorphous, non-crystalline form, contained within PLGA nanoparticles.

6. The profiled drug-coated balloon of claim 5, wherein the drug carrying nanoparticle coating consists of PLGA nanoparticles with a combination of sirolimus and paclitaxel.

7. The profiled drug-coated balloon of claim 2, wherein the distal and proximal balloon end portions are configured to expand more rapidly than the central balloon portion.

8. The profiled drug-coated balloon of claim 2, further comprising a catheter body defining said at least one inflation lumen and a central guidewire lumen.

9. The profiled drug-coated balloon of claim 8, wherein the catheter body further defines separate inflation lumens for the balloon end portions and the balloon central portion.

10. A method of intravascular drug delivering using a drug-coated balloon, comprising:
providing a profiled balloon with enlarged distal and proximal end portions and a reduced-diameter central portion, wherein a drug carrying nanoparticle coating is provided on said reduced-diameter central portion;
positioning the profiled balloon at an intravascular treatment site with said balloon in a deflated state;
inflating the enlarged distal and proximal end portions to contact the vascular wall bounding the treatment site; and
inflating the reduced-diameter central portion after the enlarged distal and proximal end portions contact the vascular wall.

11. The method of claim 10, further comprising maintaining the inflation of the reduced-diameter central portion through plural inflation and deflation cycles.

12. The method of claim 11, further comprising applying cycling pressure to the reduced-diameter central portion to create undulating pressure waves across the treatment site bounded by the enlarged distal and proximal balloon portions.

13. The method of claim 10, further comprising overinflating the reduced-diameter central portion to form a convex wall section.

14. The method of claim 10, further comprising deflating and removing the balloon without first removing any excess drug compositions surrounding the balloon.

15. A method of intravascular drug delivering using a drug-coated balloon, comprising:
providing a profiled balloon with enlarged distal and proximal end portions and a reduced-diameter central portion, wherein a drug carrying nanoparticle coating is provided on said reduced-diameter central portion;
positioning the profiled balloon at an intravascular treatment site with said balloon in a deflated state;
inflating the enlarged distal and proximal end portions to contact the vascular wall bounding the treatment site;
inflating the reduced-diameter central portion after the enlarged distal and proximal end portions contact the vascular wall;
overinflating the reduced-diameter central portion to form a convex wall section;
applying cycling pressure to the reduced-diameter central portion to create undulating pressure waves across the treatment site bounded by the enlarged distal and proximal balloon portions; and
deflating and removing the balloon without first removing any excess drug compositions surrounding the balloon.
